(19) United States
(12) Patent Application Publication
(10) Pub. No.: US 2024/0382549 A1
(43) Pub. Date: Nov. 21, 2024

(54) USE OF MULBERRY EXTRACT FOR IMPROVING SLEEP QUALITY AND/OR SUBSEQUENT BEHAVIOURAL OUTCOMES

(71) Applicant: SOCIETE DES PRODUITS NESTLE S.A., Vevey (CH)

(72) Inventors: FRANCOIS-PIERRE MARTIN, Vuisternens-devant-Romont (CH); CHRISTIAN DARIMONT-NICOLAU, Lausanne (CH); KONSTANTINOS MANTANTZIS, Renens VD (CH); CELIA FRANCEY, Berne (CH); NORMAN MOULLAN, St-Prex (CH)

(21) Appl. No.: 18/693,789

(22) PCT Filed: Sep. 21, 2022
(86) PCT No.: PCT/EP2022/076159

§ 371 Date: Mar. 20, 2024

(30) Foreign Application Priority Data
Sep. 21, 2021 (EP) 21198084.2

(51) Int. Cl. A61K 36/605; A61P 25/00

(52) U.S. Cl. CPC A61K 36/605; A61P 25/00

(57) ABSTRACT

The present invention generally relates to the use of a mulberry extract to improve sleep quality and/or subsequent behavioural outcomes. More particularly, the present disclosure relates to administration of a supplement comprising a mulberry extract, at a predetermined time before consumption of a meal and/or concurrently with consumption of a meal. Preferably the meal is a balanced evening meal.

The present invention generally relates to the use of a mulberry extract to improve sleep quality and/or subsequent behavioural outcomes. More particularly, the present disclosure relates to administration of a composition comprising the mulberry extract, at a predetermined time before consumption of a meal and/or concurrently with consumption of a meal. The combination of the composition and the meal has a glycemic load lower than the glycemic load of the meal by itself. For example, the combination of the composition and the meal has a glycemic load that is about 0 to 45. Preferably the meal is a balanced evening meal.

### BACKGROUND

Sleep is a crucial biological function and is considered an important driver of health and well-being across the lifespan. Good sleep quality has been associated with benefits for brain functions, mood and mental performance, cardio-metabolic health, as well as immunity, (Alvarez et al., 2004) whilst poor sleep quality can lead to negative consequences for health and well-being (Hublin et al., 2007).

Typical sleep architecture is comprised of two components: non-rapid eye movement (NREM; Slow Wave Sleep—SWS) and rapid eye movement (REM) sleep. Overall, sleep quality is thought to be driven by the total duration of SWS, whereas both SWS and REM have been found to jointly contribute to next-day benefits, such as improved cognition. SWS and REM are associated with distinct physiological states, including different requirements in nocturnal energy metabolism, substrate oxidation and blood glucose management.

Sleep quality is strongly associated with cognitive functioning, mood, and feelings of vitality and energy the next day. From a scientific perspective, sleep has been consistently linked with cognitive and mood benefits in humans (for reviews, see Palmer & Alfano, 2017; Rasch & Born, 2013; Walker, 2009). Among the different sleep stages, it has been suggested that SWS duration is more closely linked to declarative memory, whereas REM sleep underlies the ability to synthesize abstract information such as detecting patterns in newly acquired information (non-declarative; Rasch & Born, 2013; Walker, 2009). More recent views on the role of each sleep stage have suggested that SWS and REM might have complementary roles in the consolidation of newly acquired information (for different theories, see Rasch & Born, 2013).

The majority of evidence on the role of sleep for next-day performance comes from sleep deprivation studies, with evidence suggesting that both sleep disruption and sleep deprivation can negatively impact aspects of cognition, including declarative memory, memory encoding and recall, and cognitive flexibility in using existing information to combine them in novel ways (Walker, 2009). Among the cognitive domains strongly influenced by sleep, levels of daytime vigilance and subjective alertness are highly correlated with sleep duration (Jewett et al., 1999). In fact, vigilance tasks have been consistently used as highly sensitive measures of sleep loss (Basner & Dinges, 2011). Furthermore, sleep difficulties can have significant negative effects on emotion regulation through a range of mechanisms, including reduced ability to down regulate amygdala activation to negative information. Specifically, studies have found that sleep deprivation can cause an increase of almost 60% in amygdala activity when participants are presented with negative images (review, Palmer & Alfano, 2017).

The association between nocturnal glycemia and next-day benefits is less well-understood. Experimentally induced nocturnal hypoglycemia during deep sleep, achieved through insulin infusion to stabilize blood glucose levels to 2.2 mmol/L (40 mg/dL), has been associated with worse memory the next day (Jauch-Chara et al., 2007). In a similar manner, other studies manipulating blood glucose levels to remain within the range of 2.3-2.7 mmol/L (42-48 mg/dL) during deep sleep have uncovered lower levels of well-being, conceptualized as self-reported vitality and contentment (minor symptom evaluation profile; King et al., 1998). It should be noted that most studies linking nocturnal glycemia with cognitive/mood benefits have been conducted with diabetic patients. Therefore, there is a significant gap in understanding how nocturnal glycemia is associated with next-day benefits in healthy populations.

To date, no clear evidence exists on the role of evening meal composition for next-day benefits, and the mechanisms through which it might affect subjective and objective cognitive performance and mood.

### SUMMARY

There is a lack of causality studies on blood glucose, carbohydrate metabolism and sleep. The mechanisms underlying the link between evening dietary carbohydrates and sleep quality are yet unclear. Few studies have reported associations between glucose tolerance and sleep parameters under controlled dietary or therapeutical glucose management conditions, and most studies have used fasting parameters to explore the relationship between sleep parameters and glycemic traits.

Overall, while the knowledge on direct effect of nutrients on the brain and their mode of action to promote sleep is developed, there are still some scientific gaps on the impact of dietary deficiencies vs. supplementation for sleep quality. There is no product showing a link between improved glucose control and better sleep. Therefore, the inventors explored the relationship between nocturnal glucose metabolism, sleep quality and next-day benefits, to better define the nocturnal glucose/carbohydrate profile through a clinical study with state-of-the-art sleep and metabolic measures in healthy adults.

As set forth in greater detail later herein, the inventors identified nutritional solutions for consumption in the evening to thereby promote sleep quality, based on novel scientific evidence on evening macronutrient composition and sleep health. In particular, emerging science has shown the importance of dietary protein and carbohydrate profiles for sleep quality, which are mediated through nocturnal carbohydrate metabolism and brain functions involved in sleep-wake cycle.

A low glycemic index (GI) and fiber-rich evening diet, or a reduced glycemic response to evening meals (e.g., a reduction of at least 10%, preferably 20%, more preferably 30% in glycemic response, with a glycemic load (GL) of the evening meal), will promote better sleep quality and next day benefits in general population with sleep complaints. Yet today, the mechanism of actions remains poorly understood. One identified mode of action may relate to how high glycemic response to evening meals may result in perturbation of nocturnal glucose and carbohydrate metabolism, which can decrease sleep quality. Postprandial hyperglycemia from high dietary glycemic load and resultant compensatory hyperinsulinemia can lower plasma glucose to concentrations that compromise brain glucose (3.8 mmol/L; 68 mg/dL), triggering the secretion of autonomic counterregulatory hormones such as adrenaline, cortisol, glucagon, and growth hormone. Symptoms of counter-regulatory hormone responses can include heart palpitations, tremor, cold sweats, anxiety, irritability, and hunger. In addition, hypoglycemic events have been shown to cause arousals and substantially reduce sleep efficiency even in healthy adults (Gais et al., 2003).

Accordingly, in a non-limiting embodiment, the present invention provides a method of improving sleep quality and/or subsequent behavioural outcomes. It also provides a method of treating, preventing, and/or reducing at least one of risk, incidence or severity of at least one condition for which improved sleep quality is beneficial. The method comprises orally administering a composition comprising mulberry extract (ME) to an individual, at a predetermined time before consumption of a meal and/or concurrently with consumption of a meal. The combination of the composition and the meal has a glycemic load lower than the glycemic load of the meal by itself. For example, the combination of the composition and the meal has a glycemic load that is about 0.0 to about 45.0, preferably about 11 to about 45, preferably about 20 to about 45 and lower than that of the meal by itself.

In any embodiment disclosed herein, preferably the meal is an evening meal, for example a balanced evening meal. Preferably the total amount of the mulberry extract is effective to promote better sleep quality to an individual.

In some embodiments, the composition comprises a mulberry leaf extract (MLE).

In some embodiments, the composition optionnaly further comprises an ingredient that lowers glycemic response. Preferably, the ingredient that lowers glycemic response is one or more of tryptophan (e.g., as a free amino acid and/or in a protein such as whey protein), a glucosidase inhibitor such as 1-deoxynojirimycin (DNJ) (e.g., isolated or in a mulberry leaf or fruit extract) or phloridzin (e.g., isolated or in an apple extract), arginine-proline (AP) dipeptide (e.g., isolated or in a milk protein hydrolysate), a fiber, a resistant starch, a beta-glucan, A-cyclodextrin, glucosidase (e.g., isolated and/or as part of a composition such as mulberry leaf extract), a polyphenol (such as anthocyanins), or an amylase inhibitor (e.g., isolated and/or in a composition such as white kidney bean or wheat albumin).

In an embodiment the composition is administered to the individual in a serving that provides about 50 mg to about 2 g of ME per serving, for example about 100 mg to about 2 g ME/serving, preferably about 100 mg to about 1 g ME/serving, such as about 100 mg to about 500 mg ME/serving, such as about 250 mg ME/serving. Optionally the total amount of the ME in the composition and any of the ME in the meal is effective to promote better sleep quality to an individual.

The ME can have a concentration of DNJ at least 1% wt/wt (dry weight), least 2% wt/wt, at least 3% wt/wt, at least 4% wt/wt, for example about 5% wt/wt. In another embodiment the ME can have a concentration of DNJ at least 5% wt/wt, for example at least 6% wt/wt. In an embodiment, the composition comprising ME comprises at least 1 mg DNJ per g dry weight of the composition, preferably at least 2 mg DNJ, preferably at least 3 mg DNJ, preferably at least 4 mg DNJ, more preferably at least 5 mg DNJ, such as at least 6 mg DNJ per g dry weight of the composition.

In an embodiment the composition is administered to the individual in a serving that provides about 1 mg to about 100 mg of DNJ per serving, for example about 5 mg to about 50 mg DNJ/serving, preferably about 10 mg to about 30 mg DNJ/serving, or about 10 mg to about 20 mg DNJ/serving such as about 10 mg to about 15 mg ME/serving, such as about 12.5 mg DNJ/serving.

In an embodiment, the composition comprising ME further comprises a soluble fibre. In a preferred embodiment the soluble fibre is selected from a polydextrose or a resistant starch. In an embodiment the composition comprises ME and soluble fibre in a ratio fibre: ME of from about 1:1 to about 20:1, preferably from about 4:1 to about 10:1

The inventors recognized that whole meal replacements to be consumed everyday may result in low consumer compliance to improve sleep. Instead, a particularly advantageous embodiment disclosed herein provides a composition (e.g., a food, a beverage such as a beverage powder or a liquid beverage, or a supplement) to consume with an evening meal, and the composition reduces the glycemic response to the evening meal to thereby promote sleep quality. The compositions and methods disclosed herein can improve sleep quality by improving nocturnal glycemia during the first hours of sleep (e.g., slow wave sleep (SWS)) which is paramount for promoting the restorative benefits of sleep.

In a particular non-limiting embodiment, the present disclosure provides a product that is a low caloric, optionally low volume (preferably about 100 mL to 250 mL) nutritional solution, and the product comprises ME. lowering glycemic response to evening meals to thereby promote sleep quality, (ii) optionally a protein source rich in bioavailable tryptophan to thereby promote sleep quality, and (iii) optionally one or more supporting ingredients contributing to sleep initiation.

In some embodiments, the product is provided as a dairy powder stick to be reconstituted in a water/dairy diluent, or provided as a powdered product or RTD, or as a plant-based beverage; and the product is orally consumed together with a standardized evening meal. The product and the meal can be consumed between about three (3) hours before bedtime and at least about four (4) hours before bedtime.

Additional features and advantages are described in, and will be apparent from, the following Detailed Description and the Figures.

### BRIEF DESCRIPTION OF DRAWINGS

FIGS. 1a-d show postprandial glucose excursion by adding MLE before or mixed within a meal. FIG. 1a is a graph showing postprandial glucose excursion (in mM) over time (in min) for the 3 groups: water 5 min before a standardized balanced meal “Control (white dots)”; MLE diluted in water, consumed 5 min before a standardized balanced meal “MLE Before (grey triangles)”; and MLE consumed with a standardized balanced meal “MLE During (black dots).” FIG. 1b is a graph showing 2 h-incremental area-under-the-curve (iAUC 2 h) of the three groups. FIG. 1c is a graph showing incremental maximal glucose concentration (iCmax) in mM of the three groups. FIG. 1d is a graph showing time at which maximum postprandial glucose response is reached (min). All data is presented as means and standard error of mean (SEM). Asterisks (*) denote significant differences between control and intervention groups (p<0.05), while $ denotes significant difference (p<0.05) between the “Before” and “During” groups.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. Ranges defined using “between” include the referenced endpoints.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.” Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment or component thereof comprises more than 50 wt. % of the individually identified components, preferably at least 75 wt. % of the individually identified components, more preferably at least 85 wt. % of the individually identified components, most preferably at least 95 wt. % of the individually identified components, for example at least 99 wt. % of the individually identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal benefitting from reduced postprandial glucose. While the term “individual” or “subject” is often used herein to refer to a human, the present disclosure is not so limited. Accordingly, the term “individual” or “subject” refers to any animal, mammal or human that can benefit from the methods and compositions disclosed herein.

The relative terms “improve,” “promote,” “enhance” and the like refer to the effects of the method disclosed herein on sleep quality, particularly the administration of a composition comprising a mulberry extract, at a predetermined time before consumption of an evening meal and/or concurrently with consumption of an evening meal, relative to consumption of an identically formulated meal but without the mulberry extract provided by the composition. In some embodiments, sleep quality can be quantified by one or both of (a) a total duration of slow wave sleep (SWS) and/or (b) a total duration of rapid eye movement (REM). For example, an improved sleep quality can be established by one or both of a longer total duration of SWS and/or a total duration of REM. In some embodiments, improvement in sleep quality is improvement of one or more of i) sleep efficiency (e.g. measured by actigraphy data); ii) change in sleep latency (e.g actigraphy data); iii) change in wake after sleep onset (e.g. by actigraphy); iv) change in total sleep duration (mins, actigraphy); v) time in bed; vi) minutes spent in bed after waking up. In other embodiments, sleep quality may be assessed by self reporting (e.g. Karolinska Sleepiness Scale (KSS) or Epworth Sleepiness Scale (ESS).

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional. The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder.

As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual. As used herein, “administering” includes another person providing a referenced composition to an individual so that the individual can consume the composition and also includes merely the act of the individual themselves consuming a referenced composition.

The terms “food,” “food product” and “food composition” mean a composition that is intended for ingestion by an individual, such as a human, and that provides at least one nutrient to the individual. “Food” and its related terms include any food, feed, snack, food supplement, treat, meal substitute, or meal replacement, whether intended for a human or an animal. The food supplement can be an oral nutritional supplement (ONS), it can be can be in a form of a solid powder, a powdered stick, a capsule, or a solution. Animal food includes food or feed intended for any domesticated or wild species. In preferred embodiments, a food for an animal represents a pelleted, extruded, or dry food, for example, extruded pet foods such as foods for dogs and cats.

Within the context of the present disclosure, the term “beverage,” “beverage product” and “beverage composition” mean a potable liquid product or composition for ingestion by an individual such as a human and provides water and may also include one or more nutrients and other ingredients safe for human consumption to the individual.

The terms “serving” or “unit dosage form,” as used herein, are interchangeable and refer to physically discrete units suitable as unitary dosages for human and animal subjects, each unit containing a predetermined quantity of the composition comprising an ingredient that lowers glycemic response as disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. The term “additional” ingredient for the compositions disclosed herein does not necessarily imply that the meal consumed with the composition includes a portion of the ingredient that lowers glycemic response; instead, some embodiments of the meal consumed with the composition include a portion of the ingredient that lowers glycemic response, and some embodiments of the meal consumed with the composition lack the ingredient that lowers glycemic response in some embodiments.

### EMBODIMENTS

An aspect of the present disclosure is a method of improving sleep quality and/or subsequent behavioural outcomes. The method comprises orally administering a composition comprising mulberry extract to an individual, at a predetermined time before consumption of a meal (e.g., about thirty minutes before the meal to about one hour before the meal) and/or concurrently to consumption of a meal by the individual. If the composition comprising the mulberry extract is administered before the meal, it can be delivered in a form (e.g. capsule, liquids) that allows the composition to be present in the gastrointestinal tract at the same time the carbohydrate is/are digested.

The combination of the composition and the meal has a glycemic load lower than the glycemic load of the meal by itself. For example, the combination of the composition and the meal has a glycemic load that of the meal and is about 11 to about 45, preferably about 20 to 45.

Preferably the meal is an evening meal.

Preferably the total amount of the mulberry extract in the composition and any mulberry extract in the balanced meal is effective to promote better sleep quality to the individual.

Subsequent behavioural outcomes enhanced by improved sleep quality include one or more of (a) less frequent and/or less severe sleepiness, stress, tension/anxiety, fatigue/inertia or depression/dejection, anger/hostility, subjective frustration and/or (b) more and/or better sleep initiation, relaxation, calmness, alertness, vigor/activity, friendliness, cognition, memory, working memory, attention, vigilance, processing speed, fat utilization, weight management, immunity, subjective perceptions of mental, physical, temporal demands, subjective performance perception or next-day mood.

In another embodiment, the present disclosure provides a method of treating, preventing, and/or reducing at least one of risk, incidence or severity of at least one condition for which improved sleep quality is beneficial. The method comprises orally administering a composition comprising mulberry extract (ME) to an individual, at a predetermined time before consumption of a meal (e.g., about thirty minutes before the meal to about one hour before the meal) and/or concurrently to consumption of a meal by the individual. The combination of the composition and the meal has a glycemic load lower than the glycemic load of the meal by itself. For example, the combination of the composition and the meal glycemic load that of the meal and is about 11 to about 45, preferably about 20 to about 45.

In any embodiment disclosed herein, preferably the meal is an evening meal, for example a balanced evening meal.

The meal has a glycemic load of about 26.0 to about 58.5, for example at least about 27.0, at least about 28.0, at least about 29.0, at least about 30.0, at least about 31.0, at least about 32.0, at least about 33.0, at least about 34.0, at least about 35.0, at least about 36.0, at least about 37.0, at least about 38.0, at least about 39.0, or at least about 40.0. In some embodiments, the glycemic load of the meal is no greater than about 58.0, no greater than about 57.0, no greater than about 56.0, no greater than about 55.0, no greater than about 54.0, no greater than about 53.0, no greater than about 52.0, no greater than about 51.0, no greater than about 50.0, no greater than about 49.0, no greater than about 48.0, no greater than about 47.0, no greater than about 46.0, or no greater than about 45.0.

The combination of the composition and the meal has a glycemic load lower than the glycemic load of the meal by itself and in the range of about 11.0 to about 45.0, for example about 20 to about 45.0, for example at least about 21.0, at least about 22.0, at least about 23.0, at least about 24.0, at least about 25.0, at least about 26.0, at least about 27.0, at least about 28.0, at least about 29.0, or at least about 30.0. In some embodiments, the glycemic load of the combination of the composition and the meal is no greater than about 45, no greater than about 44.0, no greater than about 43.0, no greater than about 42.0, no greater than about 41.0, no greater than about 40.0, no greater than about 39.0, no greater than about 38.0, no greater than about 37.0, no greater than about 36.0, or no greater than about 35.0.

As another example, the glycemic load of the meal can be reduced at least about 10%, for example it can be reduced at least about 20%, preferably at least about 30.0%, most preferably at least about 40.0% in the combination of the meal and the composition.

Preferably, the composition further comprises an ingredient that lowers glycemic response in the individual. Optionally the total amount of the ingredient in the composition and any of the ingredient in the meal is effective to promote better sleep quality for the individual.

In some embodiments, the ingredient that lowers glycemic response is one or more of tryptophan (e.g., as a free amino acid and/or in a protein such as whey protein), a glucosidase inhibitor such as 1-deoxynojirimycin (DNJ) (e.g., isolated or in a mulberry leaf or fruit extract) or phloridzin (e.g., isolated or in an apple extract), arginine-proline (AP) dipeptide (e.g., isolated or in a milk protein hydrolysate), a fiber, a resistant starch, a beta-glucan, A-cyclodextrin, glucosidase (e.g., isolated and/or as part of a composition such as mulberry leaf extract), a polyphenol (such as anthocyanins), or an amylase inhibitor (e.g., isolated and/or in a composition such as white kidney bean or wheat albumin).

In some embodiments, the composition is administered once a day (e.g., with the evening meal, preferably not at other meals and/or not at other times of the day) for a total duration of at least 3 days, preferably for at least one week, more preferably for at least two weeks.

In some embodiments, the composition is a beverage administered to a human adult with sleep complaints. In some embodiments, the composition is a cereal snack, a beverage (e.g., RTD beverage) containing cereal, a soup, a porridge, a broth, or flan and is administered to a human adult. In some embodiments, the composition is administered to a human toddler.

In an embodiment the composition is administered to the individual in a serving that provides about 50 mg to about 2 g of ME per serving, for example about 100 mg to about 2 g ME/serving, preferably about 100 mg to about 1 g ME/serving, such as about 100 mg to about 500 mg ME/serving, such as about 250 mg ME/serving. Optionally the total amount of the ME in the composition and any of the ME in the meal is effective to promote better sleep quality to an individual.

The ME can have a concentration of DNJ at least 1% wt/wt (dry weight), least 2% wt/wt, at least 3% wt/wt, at least 4% wt/wt, for example about 5% wt/wt. In another embodiment the ME can have a concentration of DNJ at least 5% wt/wt, for example at least 6% wt/wt. In an embodiment, the composition comprising ME comprises at least 1 mg DNJ per g dry weight of the composition, preferably at least 2 mg DNJ, preferably at least 3 mg DNJ, preferably at least 4 mg DNJ, more preferably at least 5 mg DNJ, such as at least 6 mg DNJ per g dry weight of the composition.

In an embodiment the composition is administered to the individual in a serving that provides about 1 mg to about 100 mg of DNJ per serving, for example about 5 mg to about 50 mg DNJ/serving, preferably about 10 mg to about 30 mg DNJ/serving, or about 10 mg to about 20 mg DNJ/serving such as about 10 mg to about 15 mg ME/serving, such as about 12.5 mg DNJ/serving.

Research has shown that mulberry plant extracts comprise various physiologically active components including flavonoids, polyphenols, polysaccharides, 1-deoxynojirimycin (DNJ) identified as α-glucosidase inhibitor, fagomine, GABA. 1-deoxynojirimycin (DNJ) is thought to be the main active ingredient acting through potent α-glucosidase inhibition.

The mulberry extract applicable to the present invention can be of any Morus origin, including, but not limited to, White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

The mulberry extract applicable to the present invention can be derived from different parts of mulberry tree, including barks (trunk, twig or root), roots, buds, twigs, young shoots, leaves, fruits or a combination thereof. The mulberry extract can be in the form of e.g. dried powders such as dried powders milled from different parts of the tree. The starting plant material of mulberry extracts can be fresh, frozen or dried mulberry materials. The extract may be used as a liquid or dried concentrated solid. Typically, such an extract includes from at least about 1% w/v 1-DNJ.

In a preferred embodiment, the mulberry extract (ME) is a mulberry leaf extract (MLE).

Mulberry extracts can be prepared by procedures well known in the art. References in this aspect can be made to Chao Liu et al., Comparative analysis of 1-deoxynojirimycin contribution degree to α-glucosidase inhibitory activity and physiological distribution in Morus alba L, Industrial Crops and Products, 70 (2015) p309-315; Wenyu Yang et al., Studies on the methods of analyzing and extracting total alkaloids in mulberry, Lishizhen Medicine and Material Medical Research, 2008 (5); and CN104666427.

Mulberry leaf extracts are also commercially available, such from Karallief Inc, USA, ET-Chem.com, China, Nanjing NutriHerb BioTech Co., Ltd, China, or from Phynova Group Ltd.

The compositions of the present invention may typically comprise from about 1% to about 50%, including from about 2% to about 30%, such as from about 5% to about 20%, and also including from about 10% to about 15% of mulberry extract by weight of the composition.

In one embodiment of the present invention the composition comprises at least 1 mg 1-deoxynojirimycin (DNJ) per g dry weight, preferably at least 1 mg DNJ per g dry weight of the composition, preferably at least 2 mg DNJ, preferably at least 3 mg DNJ, preferably at least 4 mg DNJ, more preferably at least 5 mg DNJ, such as at least 6 mg DNJ per g dry weight of the composition. In one embodiment of the present invention the composition comprises about 1 to about 20 mg DNJ per g dry weight of the composition, preferably about 2 mg to about 10 mg DNJ per g dry weight of the composition, In one embodiment of the present invention the composition comprises about 5 to about 10 mg DNJ per g dry weight of the composition, preferably about 5 mg to about 8 mg DNJ per g dry weight of the composition, such as about 6 mg to about 7 mg DNJ per g dry weight of the composition.

In an embodiment, the composition is administered to an individual in a serving that provides at least about 1 mg DNJ, preferably at least about 5 mg DNJ per serving, more preferably at least about 10 mg DNJ per serving. In some embodiments, up to 50 mg DNJ are administered per serving of the composition. In some embodiments, up to 100 mg DNJ may be administered per serving of the composition.

The composition comprising ME according to the invention may be added to, or mixed into, the meal, or may be consumed in accompaniment to the meal. In a preferred embodiment the composition comprising ME according to the invention is in the form of a powder or granulate intended to be added to, or mixed into, the meal, preferably sprinkled onto the meal.

As used herein, “meal” refers to one or more food products consumed at substantially the same time as each other; preferably such that one or more proteins, one or more carbohydrates, one or more fats and at least one micronutrient are provided by consuming the meal; more preferably such that one or more proteins, one or more carbohydrates, one or more fats, one or more vitamins and one or more minerals are provided by consuming the meal. Preferably the meal comprises a plurality of food products. As used herein, “balanced meal” refers to meal which provides all of protein, carbohydrate, fat, vitamins and minerals, in quantities and proportions suitable to maintain health or growth of an individual. The quantities and proportions of protein, carbohydrate, fat, vitamins and minerals suitable to maintain health or growth may be determined in line with the current food and nutrition regulations, and any specific requirements of the individual, for example based on age, physical activity, and/or gender.

For example, the Food and Nutrition Board of the Institutes of Medicine (IOM) current energy, macronutrient, and fluid recommendations, recommend an acceptable macronutrient distribution range for carbohydrate (45%-65% of energy), protein (10%-35% of energy), and fat (20%-35% of energy) for active individuals. In an embodiment, the balanced meal provides 45-65% of total calories from carbohydrate, 20-35% of total calories from fat and of total calories 10-35% from protein. In an embodiment, the meal provides 200 kcal to 1,000 kcal to the individual, preferably 250 kcal to 900 kcal, more preferably 300 kcal to 850 kcal, and most preferably 350 kcal to 800 kcal.

In some embodiments, “evening meal” means a meal consumed about 1.0 hours to about 6.0 hours before the onset of sleep, preferably a meal about 2.0 hours to about 5.0 hours before the onset of sleep, more preferably a meal about 2.5 hours to about 4.5 hours before the onset of sleep, most preferably about 3.0 hours to about 4.0 hours before the onset of sleep.

In some embodiments, “evening meal” means a meal consumed at about 4:30 pm to about 11:30 pm in the geographic region where the individual is located, preferably a meal consumed at about 5:00 pm to about 11:00 pm in the geographic region where the individual is located, more preferably a meal consumed at about 5:30 pm to about 10:30 pm in the geographic region where the individual is located, most preferably a meal consumed at about 6:00 pm to about 10:00 pm in the geographic region where the individual is located.

Preferably the food or supplement according to the present invention, is orally administered to the individual in a form selected from the group consisting of a dairy beverage and a non-dairy beverage, and the unit dosage form is a predetermined amount of the beverage.

In some embodiments, the composition can be a ready to drink (RTD) beverage in a container, and the unit dosage form is a predetermined amount of the RTD beverage sealed in the container, which is opened for the oral administration. For example, the predetermined amount of the RTD beverage can comprise about 50 mg to about 2 g of ME. An RTD beverage is a liquid that can be orally consumed without addition of any further ingredients. The RTD beverage can be low caloric and/or optionally low volume (e.g., about 100 mL to about 250 mL).

In other embodiments, the method comprises forming the composition by reconstituting a unit dosage form of a powder comprising the ingredient that lowers glycemic response, in water or milk to thereby form the composition subsequently orally administered to the individual (e.g., within about ten minutes after reconstitution, within about five minutes after reconstitution, or within about one minute after reconstitution). The unit dosage form of the powder can be sealed in a sachet or other package, which can be opened for the reconstitution and subsequent oral administration. For example, the predetermined amount of the powder can comprise about 50 mg to about 2 g of ME. The beverage reconstituted from the powder can be low caloric and/or low volume (e.g., about 100 mL to about 250 mL).

In some embodiments, the unit dosage form of the composition comprising the ME can further comprise one or more of melatonin, for example as pistachio powder (e.g., about 0.1 to about 0.3 mg melatonin), Vitamins B3 and B6 (e.g., from about 15% NRV to about 2 mg), magnesium (e.g., about 40 mg magnesium), and/or zinc (e.g., from about 15% NRV to about 15 mg). In some embodiments, the composition can further comprise one or more of gamma aminobutyric acid (GABA), alpha-casozepine, or theanine.

The unit dosage form of the composition comprising the ingredient that lowers glycemic response can additionally contain excipients, emulsifiers, stabilizers and mixtures thereof. The composition may include any nutritional or non-nutritional ingredient that adds bulk, and in most instances will be substantially inert, and does not significantly negate the blood glucose benefits of the composition. The filler material most typically includes a fiber and/or carbohydrate having a low glycemic index.

Carbohydrate sources suitable for inclusion in the compositions disclosed herein include those having a low glycemic index, such as fructose and low DE maltodextrins, because such ingredients do not introduce a high glycemic load into the composition. Other suitable components of the composition include any dietary fiber suitable for human or animal use, including soluble and insoluble fiber, especially soluble fibres. Beneficial effects of soluble fibres on glucose response have been widely reported. Non-limiting examples of suitable soluble fibres include FOS, GOS, inulin, resistant maltodextrins, partially hydrolysed guar gum, polydextrose and combinations thereof.

A non-limiting example of a commercially available fiber for the composition include Sunfiber® (Taiyo International, Inc.), which is a water-soluble dietary fiber produced by the enzymatic hydrolysis of Guar beans; Fibersol 2™ (Archer Daniels Midland Company), which is a digestion resistant maltodextrin; and polydextrose.

In an embodiment, the composition may also comprise tryptophan. In a preferred embodiment, the composition comprises protein that comprises at least a portion of the tryptophan in the composition, preferably whey protein such as whey protein isolate; a mixture of whey protein and casein; or soy protein. In some embodiments, the supplement is administered in a unit dosage form comprising about 120 mg to about 5 g of the tryptophan.

In an embodiment, the composition can comprise tryptophan, a mulberry extract and a soluble fibre. In a preferred embodiment, the composition comprises a soluble fibre selected from polydextrose, a resistant maltodextrin (such as the soluble corn fiber Fibersol-2) and combinations thereof.

The composition may also comprise other filler, stabilizers, anti-caking agents, anti-oxidants or combinations thereof.

The composition may further comprise one or more additional components such as minerals; vitamins; salts; or functional additives including, for example, palatants, colorants, emulsifiers, antimicrobial or other preservatives. Non-limiting examples of suitable minerals for the compositions disclosed herein include calcium, phosphorous, potassium, sodium, iron, chloride, boron, copper, zinc, magnesium, manganese, iodine, selenium, chromium, molybdenum, fluoride and any combination thereof. Non-limiting examples of suitable vitamins for the compositions disclosed herein include water-soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8) folic acid (vitamin B9), cobalamin (vitamin B12), and vitamin C) and fat-soluble vitamins (such as vitamin A, vitamin D, vitamin E, and vitamin K) including salts, esters or derivatives thereof.

The individual may be a mammal such as a human, canine, feline, equine, caprine, bovine, ovine, porcine, cervine or a primate. Preferably the individual is a human.

All references herein to treatment include curative, palliative and prophylactic treatment. Treatment may also include arresting progression in the severity of a disease. Both human and veterinary treatments are within the scope of the present disclosure. Preferably the composition is administered in a serving or unit dosage form that comprises a therapeutically effective or prophylactically effective amount of the ingredient that lowers glycemic response.

### NON-LIMITING EXAMPLES

### Example 1

The following non-limiting example presents experimental data developing and supporting the concepts of embodiments provided by the present disclosure.

### ABSTRACT

### Introduction

Nutritional supplements were reported to decrease glucose response of a meal, including whey protein pre-meals and mulberry leaf extract (MLE). Here, the study evaluated in non-diabetic subjects if the efficacy of these two supplements could be affected by changing timing of consumption or by different whey protein structures.

### Research Design and Methods

Two randomized crossover case-controlled studies were performed. Thirty (30) healthy subjects consumed 250 mg of mulberry leaf extract (MLE) before or with a complete balanced meal. Acute postprandial glucose response (PPGR) was monitored with a continuous glucose monitoring (CGM) device.

### Results

The supplement significantly reduced glucose response of the standard meals at all consumption timepoints. Consumption of MLE with the meal resulted in a stronger decrease in PPGR as compared to taking it before the meal (iAUC−16%, p=0.03).

### Conclusions

The study confirmed that MLE pre-meals are efficient solutions for lowering glucose response of complete meals, and their efficacy can be optimized by choosing best administration timing or protein structure.

### DETAILED RESEARCH DESIGN AND METHODS

### Study Design and Subjects

The study was monocentric with a crossover, randomized and open design. The number of experimental conditions was three (see below). Subjects were randomly assigned to a sequence of a Williams Latin square that balanced position and carry-over effect to minimize potential bias. Eligible subjects were recruited following completion of a health status questionnaire and a medical screening visit. After enrollment and before starting the experimental visits, a CGM sensor was placed on the non-dominant arm of the subjects. The day before each testing visit, subjects were required to refrain from consuming alcohol and performing strenuous exercise. They were also asked not to take any medication like aspirin or supplement containing Vitamin C that may affect CGM measurements. Since subjects could test all experimental conditions using the same CGM sensor, randomization could be performed without any restriction such as blocking.

To study the glycemic response following ingestion of MLE, thirty (30) healthy volunteers aged 18 years to 45 years were recruited. Key inclusion criteria were a healthy status, a BMI between 20 and 29.9 kg/m2 and the ability to understand and sign an informed consent form. Key exclusion criteria were food allergy and intolerance to the products, smokers and contraindications to CGM sensor placement (e.g. skin hypersensitivity). Screening of potential participants was performed by the research nurses and validated by the medical responsible. The sample size was deduced from two previous studies that both reported 25% reduction in PPGR of either a rice-based standard meal or a load of 50 g of maltodextrin. Assuming similar effect sizes and variabilities, the calculated effect size was N=30 to reach a power of 80%.

### Experimental Meals

250 mg of mulberry (Morus alba) leaf extract (5% Reducose®, Phynova/DSM), containing 12.5 mg of DNJ, was consumed either before (mixed in water) or during (sprinkled over the food) a standard meal composed of 150 g of boiled white jasmine rice, 25 g of white bread, 80 g of curry sauce and 80 g of chicken breast slices. 200 ml of water was consumed before the standard meal. The macronutrient composition of the standard meal is reported in the table in Table 1

TABLE 1

| | | | | Total | Total | Total |
|---|---|---|---|---|---|---|
| | Energy | | Protein | carbohydrates | sugars | fat |
| Intervention | (kcal) | Total (g) | (% kcal) | (% kcal) | (% kcal) | (% kcal) |
| Study 1 | 318.5 | 411.0 | 6.3 | 88.8 | 54.4 | 5.1 |
| Study 2 | 508.9 | 248.0 | 19.6 | 57.0 | 3.5 | 23.7 |

### Interventions

In this study, the participants reached the research center at 8 h00 under fasting conditions on the experimental days. Glucose reading with the CGM device was performed just before and after intake of the test products, and interstitial glucose level was continuously and automatically measured every 15 minutes until up to 2 hours after the meal.

The subjects were asked to consume a standardized complete meal within 15 minutes, with one of the 3 arms:
1. Control: 200 ml of water at 5 minutes before the standard meal
2. MLE Before: 250 mg of MLE powder dissolved in 200 ml of water at 5 minutes before the standard meal
3. MLE During: 200 ml of water at 5 minutes before the standard meal, in which 250 mg of MLE powder was mixed

### Measurements

Glucose response was measured with a CGM device, measuring interstitial glucose concentration every 15 minutes. A sensor was installed on the non-dominant arm of each subject at least 24 hours before the first visit; and a reader, as well as instructions for its use, were provided. If a sensor was lost during the study, it was replaced, and the subject could resume the study with the next testing visit, at least 24 hours after sensor insertion. The sensor was removed at the end of the study by a clinical staff member.

### Statistical Analysis

The primary endpoint in these studies was the 2h-PPGR incremental Area Under the Curve (iAUC) that was calculated using the trapezoid method for each individual PPGR after the standardized meal. Additional endpoints of interest were maximal incremental glucose value (iCmax), the time to reach this value (Tmax), and all cross-sectional timepoints, every 15 minutes after T0. At the beginning of each visit, subjects scanned the sensor with the reader right before and after test product intake and the average was calculated to determine the baseline glycaemia (T0). Descriptive statistics (Mean, SEM) were tabulated and visualized. Means were compared using paired t-tests with significance level set at 5% (two-sided), following established standards. A sensitivity analysis was performed by using a mixed model to impute possible missing data and to consider potential systematic position or carry-over effects. Since none of these effects was close to reach statistical significance, this analysis is not further presented.

### RESULTS

### Baseline Characteristics

The participants (11 males, 19 females) were young (average age ± SEM: 31±1.3 years), lean (average BMI ± SEM: 22.9±0.4 kg/m2) and normoglycaemic (average fasting glucose level ± SEM: 5±0.09 mM). There were no missing visits, but 2 missing data points due to issues with the CGM sensor. None of the subjects reported any side effect of the interventions. The number of subjects to consider in the analyses was N=30.

### Glucose Response

Average PPGR parameters are tabulated for all interventions in the table in Table 2.

TABLE 2

| | iCmax | Tmax | 2h-iAUC |
|---|---|---|---|
| Control | 2.44 ± 0.14 | 59 ± 6.8 | 167 ± 11 |
| MLE Before | 1.88 ± 0.11 | 85 ± 8.6 | 131 ± 11 |
| MLE During | 1.60 ± 0.13 | 86 ± 9.8 | 110 ± 11 |

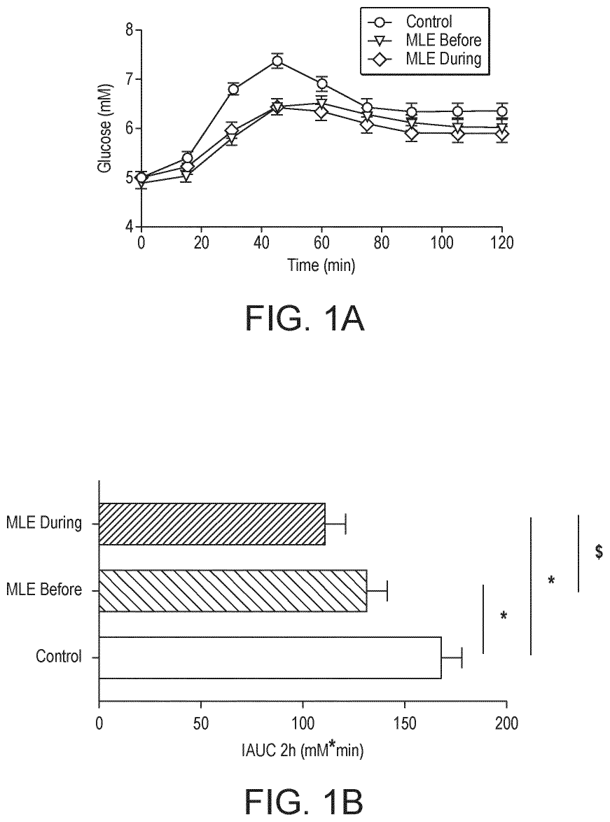

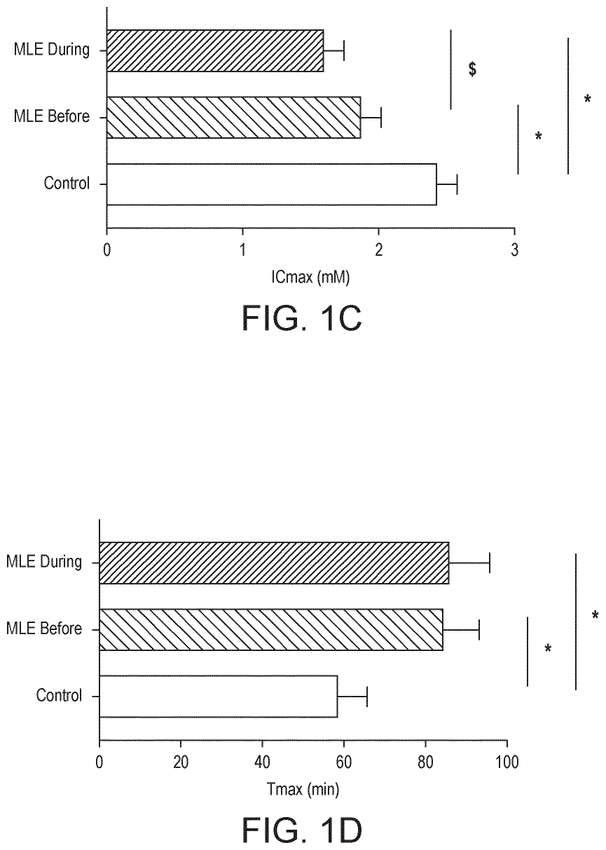

Absolute postprandial interstitial glucose values measured with CGM device in the control and 2 MLE groups are shown in FIG. 1a. Compared to the control, taking MLE before or during the meal reduced PPGR. Plots of the 2 h-iAUC (FIG. 1b) show that taking MLE before the meal significantly attenuated the glucose response by 22±7% (p=0.01), while taking MLE during the meal reduced glucose response by 34±7% (p<0.01). Interestingly, PPGR iAUC of MLE administered with the standardized balanced meal was significantly lower than postprandial glucose iAUC observed for MLE administered before the standardized balanced meal (−16±7%, p=0.03).

Comparing the maximal interstitial PPGR concentrations (FIG. 1c), the iCmax was highest in the Control arm (2.44±0.14 mM). MLE administered both just before and during the standardized meal reduced significantly iCmax of the PPGR curve compared with the control: the MLE Before group (−0.56±0.12 mM, p<0.01) and the MLE During group (−0.84±0.15 mM, p<0.01). The time to reach the maximal glucose concentration (Tmax) was earliest in the Control group (59±7 min) (FIG. 1d) and was also significantly delayed for both the MLE administered just before and during the standardized meal groups compared to the control: the MLE Before group (+26±9 min, p<0.01) and the MLE During group (+28±9 min, p<0.01). Comparing iCmax and Tmax of the group taking the MLE during versus before the standardized meal, there was a significant reduction in iCmax in the MLE During versus MLE Before group (−0.29±0.12 mM, p=0.02) but no difference in Tmax.

### Discussion

The study evaluating PPGR effects of MLE confirmed that MLE can decrease PPGR of a complete meal. Compared to an earlier study, in which the same dose of 12.5 mg DNJ (in capsule) in co-ingestion with maltodextrin resulted in 14% reduction of PPGR, the present study observed a similar reduction in PPGR of 16% when the MLE was taken in solution prior to the meal. Similarly, when MLE (8 mg DNJ) was taken with porridge, a 24% reduction of PPGR was reported. Interestingly, the present study demonstrated that timing of administration is an important aspect in obtaining optimal effects of MLE on PPGR. Indeed, MLE induced a stronger reduction of glucose response when mixed with the meal compared to ingestion before the meal. Since DNJ, the active compound in MLE, acts as a competitive α-glucosidase inhibitor, it is reasonable to expect that maximal effect will be observed when the DNJ reaches the small intestine at the same time as the carbohydrates in the food to compete for binding to the α-glucosidase enzymes. In addition to attenuating total PPGR, the present study also observed that consumption of MLE resulted in a later maximal glucose peak (later Tmax). This could mean that the MLE delays absorption of glucose in the gastrointestinal tract and possibly might stimulate GLP-1 secretion. This effect has been observed with another α-glucosidase inhibitor drug, acarbose, where delayed absorption and increased GLP-1 secretion have been demonstrated.

### Example 2

The following study explored the efficacy of nutritional interventions on sleep quality in healthy adults. This is a double-blind, controlled, randomized, 2-arm, cross-over, group sequential design clinical trial. Subjects will receive the two different nutritional interventions in a randomized order.

### Study Objectives

Primary objective; To assess the efficacy of the nutritional intervention in improving objective sleep quality among healthy adults with sleep complaints. Primary endpoints: Actigraphy parameters will be used to assess objective sleep quality: i) Change in sleep efficiency (SE), calculated as '(total time asleep/time in bed)×100; ii) Change in sleep latency (SOL), measured as the amount of time it takes subjects to fall asleep after going to bed (in minutes)

Secondary objective: To assess the efficacy of the nutritional intervention in improving subjective sleep quality. Endpoints: changes in self-reported sleep quality measured through questionnaires (e.g. Karolinska Sleepiness Scale (KSS) Total sleeping time, wake after onset (WASO).

Trial population: 45 subjects, both male and female between the ages of 25 and 50 with subjective and objective sleep complaints as measured as follows:
Subjective sleep complaints is quantified through sleep quality questionnaires (PSQI >5).
Objective sleep complaints mean sleep efficiency <85% over the 14 days of screening. For this purpose, subjects will be screened during a 2-week screening period using objective sleep monitoring devices (actigraphy).

Treatment administration: The investigational product (IP) was taken in combination with a standardized evening meal (with a glycemic load of 55), orally consumed at least 4 hours before bedtime, and within 30 minutes. The investigational product is consumed once a day for a total duration of two weeks i.e.; 14 days (28 days total for both test and control products).

Test product; beverage to be consumed during the evening meal, containing per serving:
750 mg of mulberry leaf extract containing 1% (m/m) 1-deoxynojirimycin
5.4 g of Whey protein (providing 120 mg of tryptophan)
Fortification in micronutrients: Zinc (1.337 mg), Magnesium (12.39 mg), Vitamins B3 (1.96 mg) and B6 (0.13 mg)

Control product: beverage to be consumed during the evening meal, containing per serving an equivalent protein content low in tryptophan content (4 g of gluten hydrolysate)

The test and control products are provided as powder sachets to be reconstituted in water to a final volume between 200-250 mL.

Treatment and duration: the Investigational product is to be consumed during the meal and will be administered once a day for a total duration of two weeks i.e.; 14 days (28 days total for both test and control products)

The two intervention periods will be separated by a washout period of at least 4 weeks to ensure the subjects return to their baseline sleep status and ensuring no carry over effect, and of at least 6 weeks to ensure female subjects are in the same phase of the menstrual cycle.

During the intervention period, subjects will be provided with customized meals which consist of evening meals, pre-dinner snacks and post dinner beverage. The evening meals are designed based on local dietary guidelines prepared from local commonly consumed foods with a mixture of Asian and western components. The total energy intake (TEI) is based on Estimated Energy Requirements (EER) calculated for adult male and females based on Oxford equation (Henry, 2005). A total of 4 different evening meal menus will be provided to subjects with male and female adapted serving sizes but will otherwise be standardized for macronutrient content. For the evening meals, the profile of the carbohydrates is designed to provide a glycemic load of 55±10%.

Statistical analysis: Continuous variables will be summarized using the appropriate descriptive statistics, including and not limited to: number of observations (n), mean, standard deviation (SD), median, minimum, and maximum.
Primary endpoints: The effect of the intervention on the primary sleep quality parameters (sleep efficiency and sleep latency) will be assessed though a linear mixed-effect model adjusted for the baseline values of the sleep quality parameter
Secondary endpoints: The secondary sleep quality parameters will be analyzed similarly as the primary sleep endpoints.

### RESULTS

### Sleep Efficiency and Sleep Onset Latency

We observed 81% sleep efficiency in total which is explained by a population of test-persons with sleep concerns. After treatment, we observed for sleep efficiency a statistical trend for sleep efficiency improvement (p=0.09) of 1.4% compared to control.

Moreover, Table 1 below reports “sleep onset latency” values at 4-6 days after treatment and 13-14 days of treatment, showing significant positive changes on these days.

TABLE 1
treatment difference of sleep onset latency
in (min) estimated by a mixed model.

| days | Delta | Lo | Up | p-value |
|---|---|---|---|---|
| 1-3 | 0.5 | −8.6 | 9.6 | 0.9159 |
| 4-6 | −9.8 | −18.9 | −0.7 | 0.0342 |
| 13-14 | −11.8 | −20.9 | −2.8 | 0.0112 |

### Secondary Outcomes

The point estimate of total sleeping time is positive by 3.3 minutes (p=0.8). A positive/stable total sleeping time emphasis the finding in sleep efficiency, because sleep efficiency is total sleeping time divided by total time in bed. After 13-14 days, the treatment difference of wake up after sleep onset, as well as of total time in bed were significant decreased by approx. 15 minutes (p=0.08), 50 minutes (p=0.048), respectively. (Table 2 and 3).

TABLE 2
Treatment difference of wake after sleep onset
in (min) estimated by a mixed model.

| days | Delta | Lo | Up | p-value |
|---|---|---|---|---|
| 1-3 | 0.4 | −16.1 | 16.9 | 0.96 |
| 4-6 | 0.1 | −16.4 | 16.6 | 0.99 |
| 13-14 | −14.8 | −31.3 | 1.7 | 0.078 |

TABLE 3
Treatment difference of total time in bed
in (min) estimated by a mixed model.

| days | Delta | Lo | Up | p-value |
|---|---|---|---|---|
| 1-3 | 15.4 | −34.6 | 65.3 | 0.54 |
| 4-6 | −40.6 | −90.6 | 9.3 | 0.11 |
| 13-14 | −50.5 | −100.5 | −0.6 | 0.048 |

Overall, the sleep actigraphy findings for sleep quality suggest an improvement in sleep efficiency by faster falling asleep and less wake up time during the night.

Also, results showed that the treatment statistically decreased the Karolinska Sleepiness Scale (KSS) measuring an at day sleepiness (Delta=−0.46; p=0.002) indicating an effect of product intake on the secondary effect of improved sleep quality.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of improving sleep quality and/or subsequent behavioural outcomes, the method comprising orally administering a composition comprising a mulberry extract to an individual, at a predetermined time before consumption of a meal and/or concurrently with consumption of a meal.

2. A method of treating, preventing, and/or reducing at least one of risk, incidence or severity of at least one condition for which improved sleep quality is beneficial, the method comprising orally administering a composition comprising a mulberry extract to an individual, at a predetermined time before consumption of a meal and/or concurrently with consumption of a meal.

3. The method according to any of claim 1, wheren the meal combination of the composition and the meal has a glycemic load is lower than that of the meal and about 0.0 to about 45.

4. The method according to any of claim 1, wheren the meal combination of the composition and the meal has a glycemic load from about 11 to about 45, preferably about 20.0 to about 45.0.

5. The method of any of claim 1, wherein the meal is an evening meal, preferably a balanced evening meal.

6. The method of any of claim 1, wherein the total amount of the mulberry extract in the composition and any mulberry extract in the meal is effective to promote better sleep quality to an individual.

7. The method of any of claim 1, wherein the mulberry extract is preferably mulberry leaf extract.

8. The method of any of claim 1, wherein the composition further comprises an ingredient that lowers glycemic response, wherein the ingredient that lowers glycemic response is selected from the group consisting of tryptophan, a glucosidase inhibitor, 1-deoxynojirimycin (DNJ), arginine-proline (AP) dipeptide, a fiber, a resistant starch, a beta-glucan, A-cyclodextrin, glucosidase, a polyphenol, and an amylase inhibitor.

9-12. (canceled)

13. The method of any of claim 1, wherein the composition is administered to the individual once per day for at least three days.

14. The method of any of claim 1, wherein the combination of the composition and the meal has a lower glycemic load than that of the meal by itself by at least about 10%.

15-17. (canceled)

18. The method according to claim 2, wheren the meal combination of the composition and the meal has a glycemic load is lower than that of the meal and about 0.0 to about 45.

19. The method according to claim 2, wheren the meal combination of the composition and the meal has a glycemic load from about 11 to about 45, preferably about 20.0 to about 45.0.

20. The method of claim 2, wherein the meal is an evening meal, preferably a balanced evening meal.

21. The method of claim 2, wherein the total amount of the mulberry extract in the composition and any mulberry extract in the meal is effective to promote better sleep quality to an individual.

22. The method of claim 2, wherein the mulberry extract is preferably mulberry leaf extract.

23. The method of claim 2, wherein the composition further comprises an ingredient that lowers glycemic response, wherein the ingredient that lowers glycemic response is selected from the group consisting of tryptophan, a glucosidase inhibitor, 1-deoxynojirimycin (DNJ), arginine-proline (AP) dipeptide, a fiber, a resistant starch, a beta-glucan, A-cyclodextrin, glucosidase, a polyphenol, and an amylase inhibitor.
